SEQUENCE LISTING (15 sequences)

SEQ ID NO: 1 - DNA, 31 bp, Artificial Sequence
  chemically treated genomic DNA (Homo sapiens)
ggtatattgttgaaagtaattgaataaaatc

SEQ ID NO: 2 - DNA, 30 bp, Artificial Sequence
  chemically treated genomic DNA (Homo sapiens)
aaacaataccctctaaaataaaaattaacg

SEQ ID NO: 3 - DNA, 158 bp, Artificial Sequence
  chemically treated genomic DNA (Homo sapiens)
ggtatattgttgaaagtaattgaataaaattggaaatttgagaaggtgtttgtttggattggtgagattttgaggggagaaagaagtggggattttgttggtattagtggtgttttttttttggttattgttaatttttattttagagggtattgttt

SEQ ID NO: 4 - DNA, 31 bp, Artificial Sequence
  chemically treated genomic DNA (Homo sapiens)
ggtatattgttgaaagtaattgaataaaatt

SEQ ID NO: 5 - DNA, 31 bp, Artificial Sequence
  chemically treated genomic DNA (Homo sapiens)
aaaacaataccctctaaaataaaaattaaca

SEQ ID NO: 6 - DNA, 30 bp, Artificial Sequence
  chemically treated genomic DNA (Homo sapiens)
ggtatattgttgaaagtaattgaataaaat

SEQ ID NO: 7 - DNA, 29 bp, Artificial Sequence
  chemically treated genomic DNA (Homo sapiens)
aaacaataccctctaaaataaaaattaac

SEQ ID NO: 8 - DNA, 158 bp, Artificial Sequence
  chemically treated genomic DNA (Homo sapiens)
ggtatattgttgaaagtaattgaataaaatcggaaattcgagaaggcgttcgttcggattggtgagattttgaggggagaaagaagcggggatttcgtcggtattagcggcgtttttttttcggttatcgttaatttttattttagagggtattgttt

SEQ ID NO: 9 - DNA, 31 bp, Artificial Sequence; identical to SEQ ID NO: 4
  chemically treated genomic DNA (Homo sapiens)

SEQ ID NO: 10 - DNA, 30 bp, Artificial Sequence
  chemically treated genomic DNA (Homo sapiens)
aaacaataccctctaaaataaaaattaaca

SEQ ID NO: 11 - DNA, 30 bp, Artificial Sequence; identical to SEQ ID NO: 6
  chemically treated genomic DNA (Homo sapiens)

SEQ ID NO: 12 - DNA, 29 bp, Artificial Sequence
  chemically treated genomic DNA (Homo sapiens)
aaacaataccctctaaaataaaaattaac

SEQ ID NO: 13 - DNA, 31 bp, Artificial Sequence; identical to SEQ ID NO: 1
  chemically treated genomic DNA (Homo sapiens)

SEQ ID NO: 14 - DNA, 158 bp, Artificial Sequence
  chemically treated genomic DNA (Homo sapiens)
gggcagtgccctctggaatgggggttaacggtggccgaggagggggcgccgctggtgccggcgaagtccccgcttctttctcccctcaaaatctcaccaatccgaacgaacgccttctcgaatttccgattttattcaattactttcaacaatgtgcc

SEQ ID NO: 15 - DNA, 158 bp, Artificial Sequence
  chemically treated genomic DNA (Homo sapiens)
gggtagtgttttttggaatgggggttaacggtggtcgaggagggggcgtcgttggtgtcggcgaagttttcgtttttttttttttttaaaattttattaattcgaacgaacgttttttcgaattttcgattttatttaattatttttaataatgtgtt

(19) United States
(12) Patent Application Publication
(10) Pub. No.: US 2007/0254293 A1
(43) Pub. Date: Nov. 1, 2007

(54) Method for the Analysis of Methylated Dna

(75) Inventors: David Guetig, Berlin (DE); Matthias Schuster, Berlin (DE); Juergen Distler, Berlin (DE)

Correspondence Address: KRIEGSMAN & KRIEGSMAN, 30 TURNPIKE ROAD, SUITE 9, SOUTHBOROUGH, MA, 01772, US

(73) Assignee: EPIGENOMICS AG, Berlin (DE)

(21) Appl. No.: 11/660,804

(22) PCT Filed: Aug. 9, 2005
(86) PCT No.: PCT/EP05/08792

§ 371 Date: Feb. 20, 2007

(30) Foreign Application Priority Data
Aug. 19, 2004 (EP) 04090321.3

(51) Int. Cl. C12Q 1/68

(52) U.S. Cl. 435/6

(57) ABSTRACT

The present invention relates to a method for the analysis of methylated cytosines in DNA. In the first step of the invention unmethylated cytosines in the DNA to be analysed are chemically converted into uracil while 5-methylcytosines remain unchanged. In a second step a methylation specific oligonucleotide carrying a non-extendable 3′ end is annealed to the converted DNA. Subsequently, the non-extendable 3′ terminus of the oligonucleotide is removed in case the oligonucleotide is bound to the DNA with the methylation status to be detected. Finally the unblocked oligonucleotide is extended, and the methylation status is concluded from the absence or presence of an extended oligonucleotide product. The method is preferably used for diagnosis and/or prognosis of adverse events for individuals, for distinguishing cell types and tissues, or for investigating cell differentiation.

### BACKGROUND OF THE INVENTION

The present invention relates to a method for the analysis of methylated cytosines in DNA. 5-methylcytosine is the most frequent covalent base modification in the DNA of eukaryotic cells. It plays an important biological role, e.g. in the regulation of the transcription, in genetic imprinting, and in tumorigenesis (for review: Millar et al.: Five not four: History and significance of the fifth base. In: The Epigenome, S. Beck and A. Olek (eds.), Wiley-VCH Verlag Weinheim 2003, p. 3-20). Therefore, the identification of 5-methylcytosine as a component of genetic information is of considerable interest. However, a detection of 5-methylcytosine is difficult because 5-methylcytosine has the same base pairing behavior as cytosine. As a consequence, the usual methods for identifying nucleic acids are not applicable. Moreover, the epigenetic information carried by 5-methylcytosine is completely lost during PCR amplification.

The usual methods for methylation analysis operate essentially according to two different principles. In the first case, methylation-specific restriction enzymes are utilized, and in the second case, a selective chemical conversion of unmethylated cytosines to uracil is conducted (bisulfite treatment; for review: European Patent Application 103 47 400.5, filing date: Oct. 9th 2003, applicant: Epigenomics AG). In a second step the enzymatically or chemically pretreated DNA is amplified and analyzed in different ways (for review: Fraga and Esteller: DNA methylation: a profile of methods and applications. Biotechniques. September 2002;33(3):632, 634, 636-49; WO 02/072880 pp. 1 ff).

A particularly important application of methylation analysis is the cancer diagnosis out of bodily fluids. Cancer cell DNA in bodily fluids has the property to be uniformly methylated over stretches of several 100 base pairs, while DNA of normal cells like blood shows a random mosaic methylation. However, a reliable diagnosis by detecting specially methylated cytosines in body fluids is difficult, because very small amounts of aberrant methylation patterns have to be found within a large amount of background DNA, which is methylated differently, but which has the same base sequence.

The methods known in the state of the art meet these requirements only to a certain extend. For sensitive detection the DNA is usually bisulfite treated and subsequently amplified in a methylation specific way by using either methylation specific primer or blocker oligonucleotides. The use of methylation specific primers is known as “MSP” (Herman et al.: Methylation-specific PCR: a novel PCR assay for methylation status of CpG islands. Proc Natl Acad Sci USA. Sep. 3, 1996;93(18):9821-6). The application of methylation specific blockers is known as “heavy Methyl” (WO 02/072880; Cottrell et al. A real-time PCR assay for DNA-methylation using methylation-specific blockers. Nucl. Acids. Res. 2004 32: e10). Both methods can be used as a real time PCR (”MethyLight”—WO00/70090; U.S. Pat. No. 6,331,393; Trinh et al.: DNA methylation analysis by MethyLight technology. Methods. December 2001;25(4) :456-62).

However, the applicability of these methods for the sensitive and specific detection of methylated DNA is limited. Due to the fact that an unspecific amplification of the background DNA would cause false positive results, it is necessary to increase the specificity of the amplification by using primer or blocker sequences which contain several methylation specific positions. In return, only those sequences can be analysed that comprise several CpG position. In addition, these positions have to be comethylated.

Due to these restrictions and due to the great importance of cytosine methylation there is a special technical need for methods enabling a high performance methylation analysis. In the following such a method is described.

The present invention allows a sensitive and specific detection of cytosine methylation. Herein the chemically converted DNA is amplified using specially designed oligonucleotides. Firstly, the oligonucleotides carry a methylation specific nucleotide at the 3′ end. Consequently, a mismatch free binding of the oligonucleotides only takes place with the DNA of a defined methylation status. In contrast, the DNA of the of the opposite methylation status forms a mismatch with the 3′ end of the oligonucleotides. Secondly, the nucleotide at the 3′ end of the oligonucleotides is chemically modified in a way that the extension of the oligonucleotides is blocked. In case of mismatch binding, however, the blocker nucleotide is removed by the exonuclease activity of the DNA polymerase. Subsequently, an extension of the oligonucleotide can occur. In contrast, the blocked 3′ ends of the matched oligonucleotides are not digested. As a result an extension cannot take place. In this way it is possible to selectively amplify DNA of a certain methylation state while the amplification of the background DNA is inhibited. This method allows a very sensitive and specific methylation analysis.

At the same time the scope of the present invention is different than that of the already known “MSP”- and “Heavy Methyl” methods which require comethylation of several adjacent CpG position for achieving sufficient sensitivity. Here only 1 or 2 methylation specific positions in the sequences to be analysed are targeted in a very focused manner.

A similar method for the detection of mutations is already described as “PAP” (pyrophosphorolysis-activated polymerization; e.g.: Liu and Sommer: Pyrophosphorolysis-activatable oligonucleotides may facilitate detection of rare alleles, mutation scanning and analysis of chromatin structures. Nucleic Acids Res. Jan. 15, 2002;30(2):598-604 m.w.N.; Liu and Sommer: Detection of extremely rare alleles by bidirectional pyrophosphorolysis-activated polymerization allele-specific amplification (Bi-PAP-A): measurement of mutation load in mammalian tissues. Biotechniques. January 2004;36(l):156-66 with further references.; US Patent Application 20040009515; U.S. Pat. No. 6,534,269).

The use of a special embodiment of the PAP technology, the litigation-mediated PAP (LM-PAP) for methylation analysis is mentioned in the US patent application 20040009515 (paragraph 0048 and 0179). However, the method described therein involves using methylation specific restriction enzymes. Therefore the applicability of this method is limited to certain sequences containing recognition sites of the restriction enzymes.

Here the combination of bisulfite conversion and PAP is described for the first time. This combination allows a sensitive and specific detection of cytosine methylation without the sequence dependency of LM-PAP. Since specific CpG positions are targeted rather than comethylated areas, the design of PAP-based methylation assays is comparably straightforward. The described method can be implemented to be highly sensitive to the methylation state of individual cytosines as opposed to the co-methylation requirement of MSP and Heavy Methyl. On the other hand, the described invention is also flexible enough to be adaptable to situations, were the detection of co-methylation is required. Due to the great importance of cytosine methylation and due to the above mentioned disadvantages in the prior art the present invention marks a significant technical progress.

### DESCRIPTION

The present invention provides a novel method for the analysis of cytosine methylation in DNA. The invention is characterised in that the following steps are conducted:
a) a genomic DNA sample is chemically or enzymatically treated in such a way that all of the unmethylated cytosine bases are converted to uracil or another base which is dissimilar to cytosine in terms of base pairing behaviour, while the 5-methylcytosine bases remain unchanged,
b) at least one methylation specific oligonucleotide carrying a non-extendable 3′ end is annealed to the converted DNA,
c) the non-extendable 3′ terminus of the oligonucleotide is removed in case the oligonucleotide is bound to the DNA with the methylation status to be detected,
d) the unblocked oligonucleotide is extended,
e) the methylation status is concluded from the absence or presence of an extended oligonucleotide product.

In the first step of the present invention a genomic DNA sample is chemically treated in such a way that all of the unmethylated cytosine bases are converted to uracil, or another base which is dissimilar to cytosine in terms of base pairing behaviour, while the 5-methylcytosine bases remain unchanged. Depending on the diagnostic or scientific question to be analysed the genomic DNA sample can be obtained from various sources, e.g. from cell lines, biopsies or tissue embedded in paraffin. According to the above mentioned advantages it is particularly preferred to analyse bodily fluids like plasma, serum, stool or urine. The genomic DNA is isolated by standard methods, as found in references such as Sambrook, Fritsch and Maniatis, Molecular Cloning: A Laboratory Manual, CSH Press, 2nd edition, 1989: Isolation of genomic DNA from mammalian cells, Protocol I, p. 9.16-9.19 and in the commonly used QIAamp DNA mini kit protocol by Qiagen. The conversion of unmethylated, but not methylated, cytosine bases within the DNA sample is conducted with a converting agent, preferably a bisulfite such as disulfite or hydrogen sulfite. The reaction is performed according to standard procedures (e.g.: Frommer et al.: A genomic sequencing protocol that yields a positive display of 5-methylcytosine residues in individual DNA strands. Proc Natl Acad Sci USA. Mar. 1, 1992;89(5):1827-31; Olek, A modified and improved method for bisulphite based cytosine methylation analysis. Nucleic Acids Res. Dec., 15, 1996;24(24):5064-6.; DE 100 29 915; DE 100 54 317). In a preferred embodiment, the conversion is conducted in presence of a reagent that denatures the DNA duplex and is of a radical scavenger (DE 100 29 915; German patent applications 10347397.1; 10347396.3; 10347400.5; 10347399.8; filing date: Oct. 9 2003, applicant: Epigenomics AG). It is also possible to conduct the conversion enzymatically, e.g., by use of methylation specific cytidine deaminases (German patent application 103 31 107.6, filing date: Jul. 4, 2003, applicant: Epigenomics AG).

In the second step of the present invention at least one methylation specific oligonucleotide carrying a non-extendable 3′ end is annealed to the converted DNA. A methylation specific oligonucleotide is an oligonucleotide hybridising specifically to chemically or enzymatically converted DNA which was prior to the conversion either methylated or unmethylated. As a consequence, e.g., of the bisulfite conversion unmethylated cytosine are transferred into uracil, while methylated cytosines remain unchanged. Thus, the converted DNA contains at a specific methylation position either a C (methylated cytosine) or an T/U (unmethylated DNA, first strand) or an A (unmethylated, opposing strand). Therefore, a methylation specific oligonucleotide contains at least one methylation specific C, T or A nucleotide, i.e. an nucleotide corresponding either to a methylated or unmethylated cytosine in the original, unconverted DNA.

In one preferred embodiment of the present invention the oligonucleotide is extended in case it is bound without a mismatch to the DNA to be detected. In this embodiment the 3′ end of the methylation specific oligonucleotide contains at least one C nucleotide or one methylation specific T or A nucleotide. The methylation specific nucleotide allows a mismatch free binding of the oligonucleotide to the DNA to be detected. In contrast, hybridization to the background DNA only takes place under mis-match formation. The methylation specific dinucleotide is located at the 3′ terminus. The specificity of the amplification is also affected by other factors. The influence of the length, the structure and design of the oligonucleotide, of the sequence to be detected, of the reaction compounds and the reaction conditions to the specificity of the PAP reaction is described in detail elsewhere (US Patent Application 20040009515, incorporated by reference).

The methylation specific oligonucleotide carries a non-extendable 3′ end being activatable by pyrophosphorolysis. The non-extendible 3′ terminus is a nucleotide or nucleotide analogue which has the capacity to form a Watson-Crick base pair with a complementary nucleotide and which lacks a 3′ OH capable of being extended by a nucleic acid polymerase. In one embodiment, the non-extendible 3′ terminus may be a non-extendible 3′ deoxynucleotide, such as a dideoxynucleotide. In a second embodiment, the non-extendible 3′ terminus may be a chemically modified nucleotide lacking the 3′ hydroxyl group, such as an acyclonucleotide. Acyclonucleotides substitute a 2-hydroxyethoxymethyl group for the 2′-deoxyribofuranosyl sugar normally present in dNMPs. In other embodiments, the non-extendible 3′ terminus may be other blockers as described elsewhere (US Patent Application 20040009515, incorporated by reference). Examples of a non-extendible 3′ termini include, but are not limited to, a 2′3′ -dideoxynucleotide, 3′-deoxyadenosine (cordycepin), 3′-azido-3′-deoxythymidine (AZT), 2′,3′-dideoxyinosine (ddl), 2′,3′-dideoxy-3′-thiacytidine (3TC) and 2′,3′-didehydro-2′,3′-dide-oxythymidine (d4T).

In another preferred embodiment of the present invention the oligonucleotide is extended only in case it is bound with a mismatch to the DNA to be detected. In this embodiment the 3′ end of the methylation specific oligonucleotide contains at least one non-extendible C nucleotide or one methylation specific T or A nucleotide. The methylation specific nucleotide forms a mismatch with the DNA to be detected. In contrast, hybridization to the background DNA takes place without mismatch formation. The methylation specific dinucleotide is located at the 3′ terminus.

In the third step of the present invention the non-extendable 3′ terminus of the oligonucleotide is removed in case the oligonucleotide is bound to the DNA with the methylation status to be detected. The 3′ end of the oligonucleotides is activatable by pyrophosphorolysis. This means that the blocked 3′ end can be removed by an enzyme that has pyrophosphorolysis activity in the presence of pyrophosphate (PP. sub. i) generating a nucleotide triphosphate. This activation specifically takes place when the 3′ end of the oligonucleotide is bound to the DNA without mismatch, which means only the DNA of a certain methylation status will be activated.

In a preferred embodiment DNA polymerases are used to activate the 3′termini of the oligonucleotides. Preferred DNA polymerases having pyrophosphorolysis activity are thermostable Tfl, Taq, and genetically engineered DNA polymerases, such as AmpliTaqFs and ThermoSequenase.™. These genetically engineered DNA polymerases have the mutation F667Y or an equivalent mutation in their active sites. The use of genetically engineered DNA polymerases, such as AmpliTaqFs and ThermoSequenase.™., greatly improves the efficiency of PAP (Liu and Sommer 2002, loc cit). These Family I DNA polymerases can be used when the activatable oligonucleotide is a 3′ dideoxynucleotide or an acyclonucleotide. When the activatable oligonucleotide is an acyclonucleotide, Family II archaeon DNA polymerases can also be used. Examples of such polymerases include, but are not limited to, Vent (exo-) and Pfu (exo-). These polymerases efficiently amplify 3′ acyclonucleotide blocked Oligonucleotides. Two or more polymerases can also be used in one reaction.

The activation of the 3′ end of the oligonucleotide may also occur by another mechanism, such as a 3′ exonuclease.

Other enzymes usable for the activation of the oligonucleotide (e.g. helicases, topoisomerases, telomerases) are described in detail elsewhere (US patent application 20040009515, incorporated by reference).

In the fourth step of the invention the unblocked oligonucleotide is extended. The extension is preferably conducted by a nucleic acid polymerase in the presence of nucleoside triphosphates (see above, US patent application 20040009515, incorporated by reference).

In the fifth step of the present invention the methylation status is concluded form the absence or presence of an extended oligonucleotide product. In one embodiment, the detection of the nucleic acid is performed by detecting the unblocking of oligonucleotide, e.g. by detecting the loss of a label contained in the 3′ terminal residue of the oligonucleotide. In a second aspect, the unblocking is detected by detecting the presence of a 3′ OH on the 3′ terminal residue that is capable of extension or ligation. In this aspect, the detection is determined by extending the unblocked oligonucleotide or by ligating the unblocked oligonucleotide to a further oligonucleotide. In a second embodiment, the detection of the nucleic acid is performed by detecting the extended oligonucleotide, e.g. by means of an incorporated labelled nucleotide. In a second aspect, the extended oligonucleotide is detected by gel electrophoresis. In a third aspect, the extended oligonucleotide is detected by the binding or incorporation of a dye or spectral material. The person skilled in the art knows further methods for the detection of the extended oligonucleotide. In a particularly preferred embodiment real time probes are used to detect the presence of the extension product. Several versions of real time probes are known, e.g. Lightcycler, Taqman, Scorpio, Sunrise, Molecular Beacon or Eclipse probes. Details concerning structure or detection of these probes are known in the state of the art (e.g. U.S. Pat. No. 6,331,393 with further references). For example Taqman probes can be designed by the “PrimerExpress” Software (Applied Biosystems).

In a preferred embodiment of the present invention, the PAP method described above can be performed bidirectionally (Bi-PAP). Bi-PAP uses two opposing pyrophosphorolysis activatable oligonucleotides with one nucleotide overlap at their 3′ termini. Thus, in Bi-PAP, PAP is performed with a pair of opposing activatable oligonucleotides. Both the downstream and upstream oligonucleotides are specific for the nucleotide of interest at the 3′ termini (e.g., an C:G base pair). In the initial round of amplification segments of undefined size are generated. In subsequent rounds, a segment equal to the combined lengths of the oligonucleotides minus one is amplified exponentially. Nonspecific amplification occurs at lower frequencies because this design eliminates misincorporation error from an unblocked upstream. The oligonucleotides may be 30-60 nucleotides for most efficient amplification (US patent application 20040009515, incorporated by reference).

Amplification by the PAP method can be linear or exponential. Linear amplification is obtained when the activatable oligonucleotide is the only complementary oligonucleotide used. Exponential amplification is obtained when a second opposing oligonucleotide, which may be a second activatable oligonucleotide, is present that is complementary to the desired nucleic acid strand. The activatable oligonucleotide and the second oligonucleotide flank the region that is targeted for amplification. The second oligonucleotide anneals to the separated desired nucleic acid strand product. Subsequently polymerization extends the second oligonucleotide on the desired nucleic acid strand to synthesize a copy of the nucleic acid template strand. Afterwards the synthesized nucleic acid template strand is separated from the desired nucleic acid strand. By repeating the above mentioned steps an exponential amplification can be achieved.

In a particular preferred embodiment the methylation analysis is conducted with two methylation specific oligonucleotides in combination with a methylation specific real time probe system.

The present invention also includes other modifications of PAP which are, e.g., described in detail in the US patent application 20040009515 (incorporated by reference). These modifications are—as far as they are applicable for analysis of bisulfite treated DNA—part of this invention.

The US patent application discloses ways of performing a massively parallel analysis of DNA by using microarrays. A methylation analysis by using these high throughput methods is also part of the present invention, as far as said methods are applicable for bisulfite treated DNA.

The methods disclosed here are preferably used for the diagnosis and/or prognosis of adverse events for patients or individuals, whereby these adverse events belong to at least one of the following categories: undesired drug interactions, cancer diseases, CNS malfunctions, damage or disease, symptoms of aggression or behavioural disturbances; clinical, psychological and social consequences of brain damage, psychotic disturbances and personality disorders, dementia and/or associated syndromes, cardio-vascular disease, malfunction and damage, malfunction, damage or disease of the gastrointestinal tract, malfunction, damage or disease of the respiratory system, lesion, inflammation, infection, immunity and/or convalescence, malfunction, damage or disease of the body as an abnormality in the development process, malfunction, damage or disease of the skin of the muscles, of the connective tissue or of the bones, endocrine and metabolic malfunction, damage or disease, headaches or sexual malfunction This new method also serves in a particularly preferred manner for distinguishing cell types and tissues or for investigating cell differentiation.

Part of the present invention is also a kit, which consists of methylation specific activatable oligonucleotides and optionally contains a polymerase, probes for the detection of the amplificate and/or a bisulfite reagent.

### EXAMPLE

### Example 1

### Selective Amplification of Unmethylated Connexin 26 Fragments with 3′Amino Modified Oligonucleotides Using Polymerases with Proofreading Activity

The LightCycler (Roche) is a device for conducting PCR and simultaneous detection and analysis of the PCR products. The device was modified according to the manufacturer's instructions using SybrGreen for the detection of dsDNA. The quantitative and qualitative analyses of the PCR were conducted with LightCycler Software Version 3.5. The connexin 26 fragment (accession number AF 144321.1, nt 748-905) was amplified from bisulfite treated methylated and unmethylated DNA.

The selective amplification of the unmethylated connexin 26 gene fragment was conducted in 20 μl of reaction volume (5 U of PfuI polymerase (Stratgen); 1× PfuI reaction buffer (Stratgene), 2 mM MgCl2, 200 μM of each DNTP, 300 nM of each oligonucleotide, 3′modified by C3-amino residue (oligonucleotide FIC-3NH, GGTATATTOTTGAAAGTAATTGAATAAAATC-NH2, SEQ ID 1 ; oligonucleotide RIG-3NH, AAACAATACCCTCTAAAATAAAAATTAACG-NH2, SEQ ID 2), 0.25 μg/μl BSA (Sigma, Munich), 0.25 μl of 1:1000 dilution of SybrGreen (Molecular Dynamics), 1 ng of template DNA) with the following cycler program: 95° C.-10 min; 50 cycles: denaturation 96° C.-10 s, annealing 56° C.-30 s; extension 72° C.-10 s. Detection was conducted at each amplification cycle by SybrGreen in the extension step after 10 s. The connexin 26-PCR fragment was detected monitoring the F1 channel (GGtAtATTGTTGAAAGTAATTGAATAAAATTGGAAATTTGAGAAGGTGTTTGTTTG GATTGGTGAGATTTTGAGGGGAGAAAGAAGTGGGGAtTTTGtTGGtAttAGTGGTGt ttttTttTTGGttAtTGTTAAtttttATTttAGAGGGtAtTGttt; Seq ID 3). Due to the identical base pair behavior of uracile and thymine the positions which correspond to the converted, originally unmethylated, cytosines are marked with a small “t” (resp. small “a” in the complementary strand). In contrast, capital “T” (resp. “A”) describe thymines already existing prior to bisulfite treatment.

The amplification of the connexin fragment was investigated on 1 ng methylated and unmethylated bisulfite treated DNA. Only with unmethylated DNA or mixtures of unmethylated and methylated bisulfite treated template DNA a amplification product was generated in the real time PCR. The melting point of the product was identical to that obtained with the unmodified primers (primerF2-T, GGTATATTGTTGAAAGTAATTGAATAAAATT; SEQ ID 4; primer R2-A AAACAATACCCTCTAAAATAAAAATTAACA, SEQ ID 5) with unmethylated bisulfite treated template DNA. The oligonucleotide F1C-3NH and R1-G-3NH will not be extended by polymerase without proofreading activity. PfuI shows 3′-5′exonuclease proofreading activity on primers that do not match at their 3′position with the template. The oligonucleotides F1C-3NH and R1-G-3NH show such mismatches with unmethylated bisulfite treated template and therefore the 3′-base, including the amino modification is removed. Subsequently, the remaining primer, shortened by one nucleotide acts as a primer for the PCR. Therefore, only the unmethylated connexin fragment is amplified.

### Example 2

### Selective Amplification of Methylated Connexin 26 Fragments with 3′ Modified Oligonucleotide Using Pyrophosphorolysis-Activated Exonuclease Activity of Polymerases

The LightCycler (Roche) is a device for conducting PCR and simultaneous detection and analysis of the PCR products. The device was modified according to the manufacturer's instructions using SybrGreen for the detection of dsDNA. The quantitative and qualitative analyses of the PCR were conducted with LightCycler Software Version 3.5. The connexin 26 fragment (accession number AF 144321.1 nt 748-905) was amplified from bisulfite treated methylated and unmethylated DNA.

The selective amplification of methylated connexin 26 gene fragments was conducted in 20 μl of reaction volume (5 U of FastStart Taq polymerase (Roche, Penzberg); 1× FastStart reaction buffer (Roche, Penzberg), 300 nM of each oligonucleotide, 3′modified by C3-amino residue (oligonucleotide F1C-ddC, GGTATATTGTTGAAAGTAATTGAATAAAATddC, SEQ ID 6; oligonucleotide R1G-A, AAACAATACCCTCTAAAATAAAAATTAACddg, SEQ ID 7), 0.25 μg/μl BSA (Sigma, Munich), 300 μM Na4P207 (Fluka), 0.25 μl of 1:1000 dilution of SybrGreen (Molecular Dynamics), 1 ng of template DNA) with the following cycler program: 95° C.-10 min; 50 cycles: denaturation 96° C.-10 s, annealing 56° C.-30 s; extension 72° C.-10 s. Detection was conducted at each amplification cycle by SybrGreen in the extension step after 10 s. The connexin 26-PCR fragment was detected monitoring the F1 channel (GGtAtATTGTTGAAAGTAATTGAATAAAATCGGAATTCGAGAAGGCGTTCGTTCG GATTGGTGAGATTTTGAGGGGAGAAAGAAGCGGGGAtTTCGtCGGtAttAGCGGCGt ttttTttTCGGttAtCGTTAAtttttATTttAGAGGGtAtTGttt; SEQ ID 8). The amplification of the connexin fragment was investigated on 1 ng methylated and unmethylated bisulfite treated DNA. Only with methylated DNA or mixtures of methylated and unmethylated bisulfite treated template DNA a exponential amplification of connexin was found in the real time PCR. The melting point of the product was identical to that obtained with the unmodified primers (primerF2-T, GGTATATTGTTGAAAGTAATTGAATAAAATC, SEQ ID 9; primer R2-A AAACAATACCCTCTAAAATAAAAATTAACG, SEQ ID 10) with methylated bisulfite treated template DNA.

The oligonucleotide F1C-ddC will not be extended by Fast-StartTaq polymerase without PPi in the PCR reaction. However, under PCR condition with 300 μM PPi the ddC-modified oligonucleotide gets activated by pyrophosphorolysis if the oligonucleotide matches at its 3′position with the template. The primer F1C-ddC matches to the methylated bisulfite treated template and therefore, the 3′-didesoxynucleotide is removed. Subsequently, the remaining oligonucleotide, shortened by one nucleotide, acts as a primer for the PCR. Therefore, only the methylated connexin fragment is exponentially amplified.

### Example 3

### Preferred Amplification of Non Methylated Bisulfite Treated DNA in Contrast to Methylated DNA

Human genomic lymphocyte DNA methylated with SssI methyltransferase (Chemicon) and human genomic lymphocyte DNA were bisulfite treated as described above. PCRs of a connexin promoter fragment (Accession number AL138688.27.1.127794; nt 119055 to nt119197) was performed on a Lightcycler device (Roche Diagnostics) in a total volume of 20 μl using 1 ng methylated bisulfite DNA and 1 ng unmethylated bisulfite DNA, respectively. The cycling condition were 2 min 95 C and 50 cycles of denaturation 95° C. 10 s, annealing 56° C. 30 s and extension 72° C. 30 s. The SybrGreen fluorescence was monitored in each cycle after the extension step.

The PCR conditions were as follows:

PCR A

0.6 μM forward primer (GGTATATTGTTGAAAGTAATTGAATAAAAT) (Seq ID 11), 0.3 reverse primer (AAACAATACCCTCTAAAATAAAAATTAAC) (Seq ID 12), 0.25 mg/ml bovine serum albumin, 0.2 mM dNTPs each, 2 U PfuTurbo Cx polymerase, 1× PfuTurbo reaction buffer, 3.5 mM MgCl₂, Sybrgreen 1:80000

PCR B

0.6 μM forward primer (GGTATATTGTTGAAAGTAATTGAATAAAATddC) (Seq ID 11); 0.3 reverse primer (AAACAATACCCTCTAAAATAAAAATTAAC) (Seq ID 12), 0.25 mg/ml bovine serum albumin, 0.2 mM dNTPs each, 2 U PfuTurbo Cx polymerase, 1× PfuTurbo reaction buffer, 3.5 mM MgCl, Sybrgreen 1:80000

PCR C

0.6 μM forward primer (GGTATATTGTTGAAAGTAATTGAATAAAATC-3NH) (Seq ID 13); 0.3 reverse primer (AAACAATACCCTCTAAAATAAAAATTAAC) (Seq ID 12), 0.25 Tng/ml bovine serum albumin, 0.2 mM dNTPs each, 2 U PfuTurbo Cx polymerase, 1× PfuTurbo reaction buffer, 3.5 mM MgCl₂, Sybrgreen 1:80000

Methylated and unmethylated bisulfite DNA were amplified with the same efficiency when PCR was performed according condition PCR A. In contrast, non methylated bisulfite DNA was more efficiently amplified thn methylated bisulfite DNA using the ddC or amino 3′modified primer (PCR B, PCR C conditions). The differences of the cycle thresholds are shown in table 1.

TABLE 1
Cycle threshold differences of methylated and
unmethylated template DNA amplified by different PCR conditions

| | | ΔCt of forward primer |
|---|---|---|
| PCR condition | 3′modification | (Ct methyl. − Ct unmethy. DNA) |
| A | no | −1.3 |
| B | ddC | 7.8 |
| C | NH2 | 6.2 |

PCR with DNA polymerase lacking proof reading activity failed to amplify the connexine promoter fragment with the ddC or NH2 modified forward primer (data not shown).

Sequence

Genomic sequence: 158 bp

(Seq ID 14)

Gggcagtgccctctggaatgggggttaacggtggccgaggagggggcgcc
gctggtgccggcgaagtccccgcttctttctcccctcaaaatctcaccaa
tccgaacgaacgccttctcgaatttccgattttattcaattactttcaac
aatgtgcc

Bisulfite Converted Sequence

(Seq ID 15)

Gggtagtgttttttggaatgggggttaacggtggtcgaggagggggcgtc
gttggtgtcggcgaagttttcgtttttttttttttttaaaattttattaa
ttcgaacgaacgttttttcgaattttcgattttatttaattatttttaat
aatgtgtt

1. Method for the analysis of cytosine methylation in DNA, characterised in that the following steps are conducted:
a) a genomic DNA sample is chemically or enzymatically treated in such a way that all of the unmethylated cytosine bases are converted to uracil or another base which is dissimilar to cytosine in terms of base pairing behaviour, while the 5-methylcytosine bases remain unchanged,
b) at least one methylation specific oligonucleotide carrying a non-extendable 3′ end is annealed to the converted DNA,
c) the non-extendable 3′ terminus of the oligonucleotide is removed in case the oligonucleotide is bound to the DNA with the methylation status to be detected,
d) the unblocked oligonucleotide is extended,
e) the methylation status is concluded from the absence or presence of an extended oligonucleotide product.

2. A method according to claim 1, wherein the non-extendable 3′ terminus of the oligonucleotide is methylation specific.

3. A method according to claim 1, further characterized in that the 3′ non-extendable terminus of the oligonucleotide is a nucleotide or nucleotide analogue which cannot be extended by a nucleic acid polymerase, but which can be removed by pyrophosphorolysis.

4. A method according to claim 3, further characterized in that the nucleotide or nucleotide analogue is selected from the group consisting of a 3′ deoxynucleotide, a 2′,3′-dideoxynucleotide, an acyclonucleotide, 3′-deoxyadenosine (cordycepin), 3′-azido-3′-deoxythymidine (AZT), 2′,3′-dideoxyinosine (ddl), 2′,3′-dideoxy-3′-thiacytidine (3TC) and 2′,3′-didehydro-2′,3′-dide-oxythymidine (d4T).

5. A method according to claim 1, further characterized in that the removal in step c) is performed with a DNA polymerase.

6. A method according to claim 1, further characterized in that the extension in step d) is performed with a DNA polymerase.

7. A method according to claim 1, further characterized in that in step e) the presence of an extended oligonucleotide product is detected by a label introduced by a nucleotide or nucleotide analogue present in the extension step.

8. A method according to claim 1, further characterized in that in step e) the presence of an extended oligonucleotide product is detected by the binding or incorporation of a dye or spectral material.

9. A method according to claim 8, further characterized in that in step e) the presence of an extended oligonucleotide product is detected by real time probes.

10. A method according to claim 1, further characterized in that the steps b) to d) are performed bidirectionally.

11. A method according to claim 1, further characterized in that a exponential amplification is performed by repeating the steps b) to d) for several times.

12. Use of a method according to claim 1 for the diagnosis and/or prognosis of adverse events for patients or individuals, whereby these adverse events belong to at least one of the following categories: undesired drug interactions, cancer diseases, CNS malfunctions, damage or disease, symptoms of aggression or behavioural disturbances; clinical, psychological and social consequences of brain damage, psychotic disturbances and personality disorders, dementia and/or associated syndromes, cardiovascular disease, malfunction and damage, malfunction, damage or disease of the gastrointestinal tract, malfunction, damage or disease of the respiratory system, lesion, inflammation, infection, immunity and/or convalescence, malfunction, damage or disease of the body as an abnormality in the development process, malfunction, damage or disease of the skin, of the muscles, of the connective tissue or of the bones, endocrine and metabolic malfunction, damage or disease, headaches or sexual malfunction.

13. Use of a method according to claim 1 for predicting undesired drug effects, distinguishing cell types or tissues or for investigating cell differentiation.

14. A kit, which consists of methylation specific activatable oligonucleotides and optionally contains a polymerase, probes for the detection of the amplificate and/or a bisulfite reagent.
